(19) United States
(12) Patent Application Publication
(10) Pub. No.: US 2013/0274720 A1
(43) Pub. Date: Oct. 17, 2013

(54) SPLIT ENDOSCOPIC INSTRUMENT

(71) Applicant: JAMES K. BRANNON, Leawood, KS (US)

(72) Inventor: JAMES K. BRANNON, Leawood, KS (US)

(21) Appl. No.: 13/444,559

(22) Filed: Apr. 11, 2012

(51) Int. Cl. A61B 17/00

(52) U.S. Cl. 606/1

(57) ABSTRACT

The present invention provides a split endoscopic instrument including a handle and cannula in communication with a surgical site through a lumen therein the split endoscopic instrument being comprised of separable instrument halves that meet at a parting plane and are configured for operation between open and closed configurations and further includes retaining members and interlocking connectors for securely aligning the separable instrument halves together, the handle includes circumferential grooves extending radially therearound for receiving the retaining members therein.

### BACKGROUND OF THE INVENTION

The present invention is broadly directed to improvements in instruments for endoscopic surgery and, more particularly, to a split or bivalve endoscopic instrument which is formed in separable halves.

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Some are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

In the use of endoscopic instruments, it is often necessary for the surgeon to locate the surgical site indirectly, that is, without a direct view of the site initially. This is especially true with regard to arthroscopic surgery. It has often been necessary to employ radiopaque endoscopic instruments and a radiant imaging technique, such as fluoroscopy or computed tomography to extend an endoscopic instrument from an external incision to the surgical site. It is desirable to limit irradiation of the patient if alternative procedures can be used to achieve the same objectives.

In some types of endoscopic surgery, a portal instrument or scope is used to maintain a pathway from an external incision to a surgical site, such as a hip joint. Depending on the procedures required in a given surgery, it might be necessary to repeatedly insert and remove certain endoscopic instruments or to insert and remove a sequence of such instruments. In such circumstances, it is still desirable and sometimes necessary to maintain an already established pathway from an external incision to the surgical site. Thus, it might be desirable to use a previously inserted endoscopic instrument as a guide to the next instrument which might be needed. However, in many instances the previously inserted instrument cannot be slipped completely over the subsequent instrument, thus requiring the reinsertion of a guide instrument, such as a guide wire.

### SUMMARY OF THE INVENTION

The present invention provides improvements in endoscopic surgical instrumentation by providing an endoscopic instrument formed in separable halves which can be inserted through an incision toward a surgical site, which can act as a guide for a subsequent endoscopic instrument, and which can be readily separated to facilitate use of the subsequent instrument.

An embodiment of the invention provides a bivalve or split endoscopic instrument which is formed in halves, though not necessarily equal halves, which are releasably retained together for use of the instrument in endoscopic surgery and which can be selectively separated to facilitate removal of the split endoscopic instrument from an instrument inserted through it. The embodiment of the instrument includes a proximal handle having an elongated rigid cannula extending therefrom. A lumen is formed along a longitudinal axis through the handle and the cannula from a proximal rear port of the handle to a distal tip of the cannula. The split endoscopic instrument may be provided in the form of a trephine, a trocar, an endoscopic portal instrument, or the like. Typically, a trephine has a circular saw formed at its distal tip, while a trocar has a distal tip which is cut off at an angle and sharpened, and a portal instrument has an unsharpened distal tip cut off perpendicular to the longitudinal axis of the cannula.

The embodiment of the split endoscopic instrument is formed in halves by a junction plane extending along the longitudinal axis. It is foreseen that the split endoscopic instrument components could have other proportions, such as a 60/40 percent circumferential split, a one third/two thirds split, or the like. The halves of the instrument preferably have alignment structures to properly position the joined halves relative to one another. The halves of the instrument are held together by retaining members. It is foreseen that the components of the instrument could also be formed in such a manner as to snap together. Such snap features could also form alignment structures.

In an embodiment of the split endoscopic instrument, the handle halves are provided respectively with cooperating tongues and grooves which are engaged to axially align the handle halves. Similarly, the cannula halves are provided with cooperating tabs and notches which are engaged to axially align the cannula halves and to prevent skewing of the cannula halves. It is foreseen that other types of alignment structures could be employed to facilitate joining of the instrument halves in proper alignment.

In an embodiment of the split endoscopic instrument, the handle has a larger outer diameter than the cannula. The handle is provided with a plurality of circumferential grooves which receive bands or O-rings which function as retaining members to retain the halves of the instrument together. The bands or O-rings may be formed of a resilient rubber or polymer to facilitate assembly and removal. An outer surface of the handle may be provided with a non-slip surface feature, such as knurling. The handle may be provided with an anvil surface which is recessed from the outer surface of the handle and which intersects the retaining members to facilitate separation of the instrument halves by severing the retaining members, as with a scalpel, scissors, or the like. The split endoscopic instrument may be formed of any surgically acceptable material, such as a stainless steel, a polymer, or the like.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is an enlarged side elevational view of an embodiment of a split endoscopic instrument according to the present invention.

FIG. 2 is a plan view of disassembled components of the split endoscopic instrument, illustrated at a somewhat reduced scale.

FIG. 3 is a plan view of the disassembled components of the split endoscopic instrument, illustrating inner sides of halves of the instrument.

FIG. 4 is a greatly enlarged fragmentary plan view of the inner sides of the instrument halves and illustrates internal alignment structures formed at a proximal end of the instrument.

FIG. 5 is a view similar to FIG. 4 and illustrates internal alignment structures formed at a distal end of the instrument.

FIG. 6 is a greatly enlarged elevational view of a grip section of the instruments, shown with one of the retaining members removed.

FIG. 7 is a greatly enlarged elevational view of the grip section of the split endoscopic instrument and illustrates a recessed anvil surface to facilitate severing of the retaining members to enable separation of the halves of the instrument.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a split endoscopic instrument according to the present invention. The instrument 1 generally includes a handle 2 and an elongated cannula 3 which are formed in separable components, such as half instrument units or halves 4 and 5 which are retained together by retaining members 6.

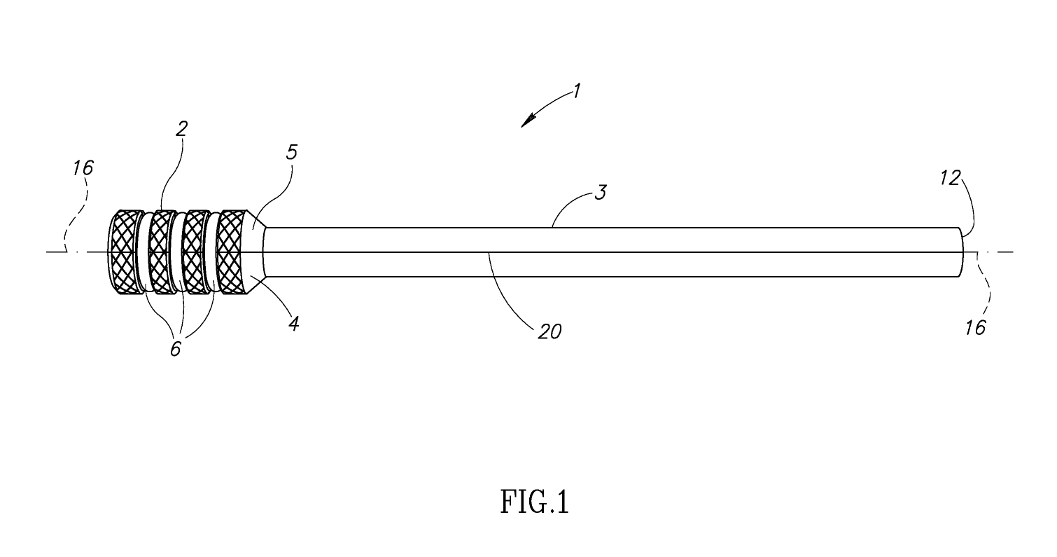

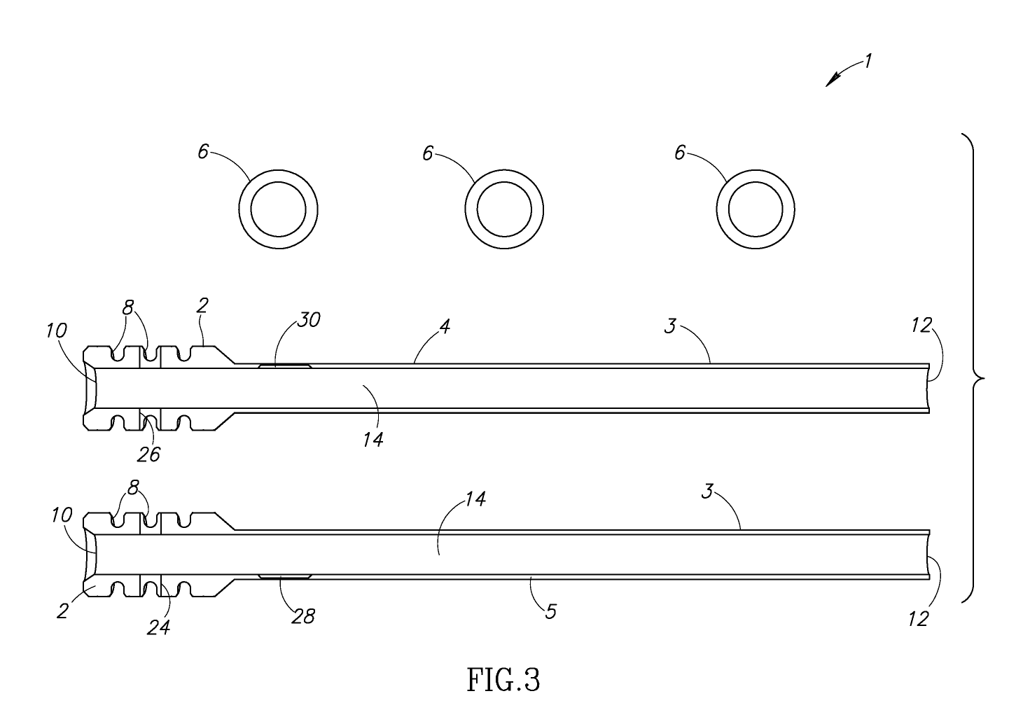

The handle 2 of the illustrated instrument is radially enlarged relative to the cannula 3 and has a non-slip surface feature such as knurling. Circumferential grooves 8 are formed into the handle 2 to receive the retaining members 6. The handle 2 terminates proximally in a rear port 10 (FIG. 3), while the cannula 3 terminates distally in a cannula tip 12. A cylindrical lumen 14 extends from the rear port 10 through the handle 2 and the cannula 3 to the distal cannula tip 12 and defines a longitudinal axis 16 (FIGS. 1, 6, and 7) of the instrument 1. The illustrated instrument 1 is formed in the halves 4 and 5 which are joined generally along a junction or parting plane 20 passing through the longitudinal axis 16. It is foreseen that the split endoscopic instrument could alternatively be formed in a manner other than in halves, and the present invention is not intended to be limited to any particular proportions of the separable instrument components 4 and 5, thus the parting plane 20 does not necessarily define equal halves 4 and 5.

The illustrated endoscopic instrument 1 is configured as an endoscopic portal instrument for establishing and maintaining a path from an external incision in a patient to a surgical site. However, it is foreseen that improvements of the present invention could be applied to other endoscopic instruments, such as trephines, trocars, and the like. Typically, portal instruments are used for inserting other endoscopic instruments toward the surgical site and sometimes for managing the exchange of fluids with the surgical site. The instrument 1, as illustrated, would not be suitable for fluid management because of possible leakage along the parting plane 20. However, it is foreseen that mating portions of the instrument 1 at the parting plane 20 could be provided with seals (not shown) to prevent leakage of fluids from the instrument 1. It is also foreseen that the handle 2 could be provided with a side port (not shown) in fluid communication with the lumen 14.

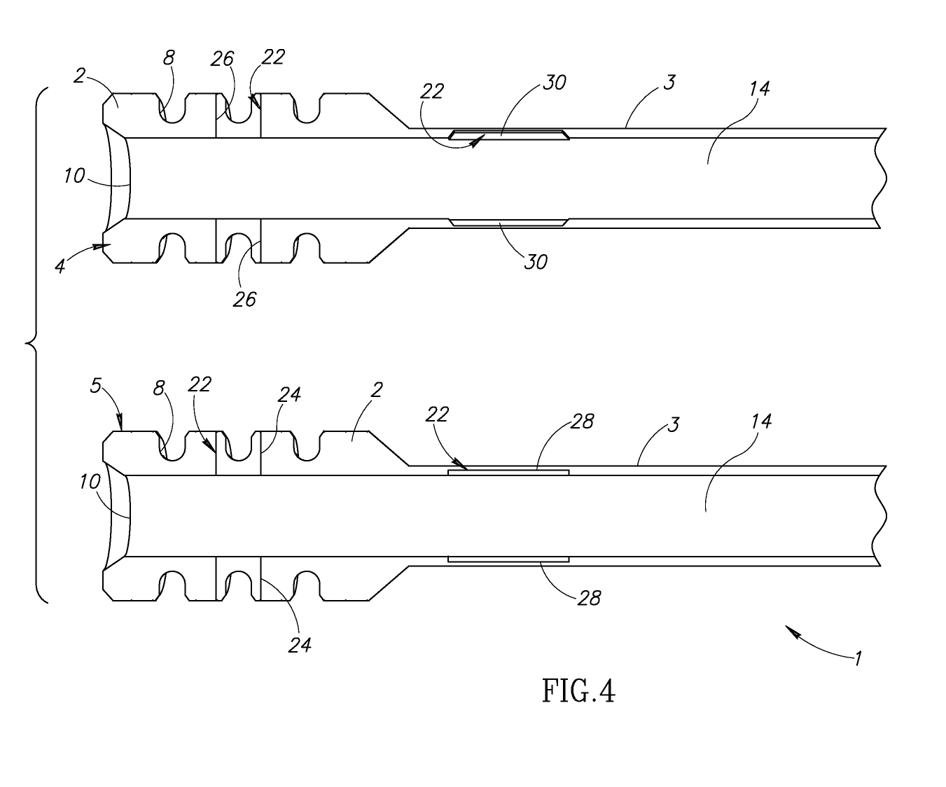

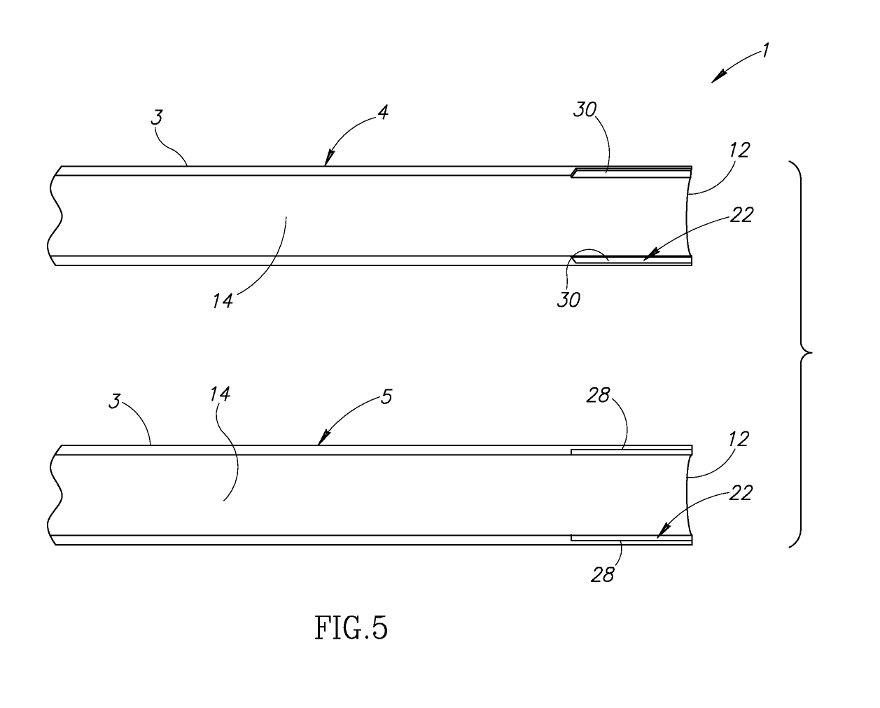

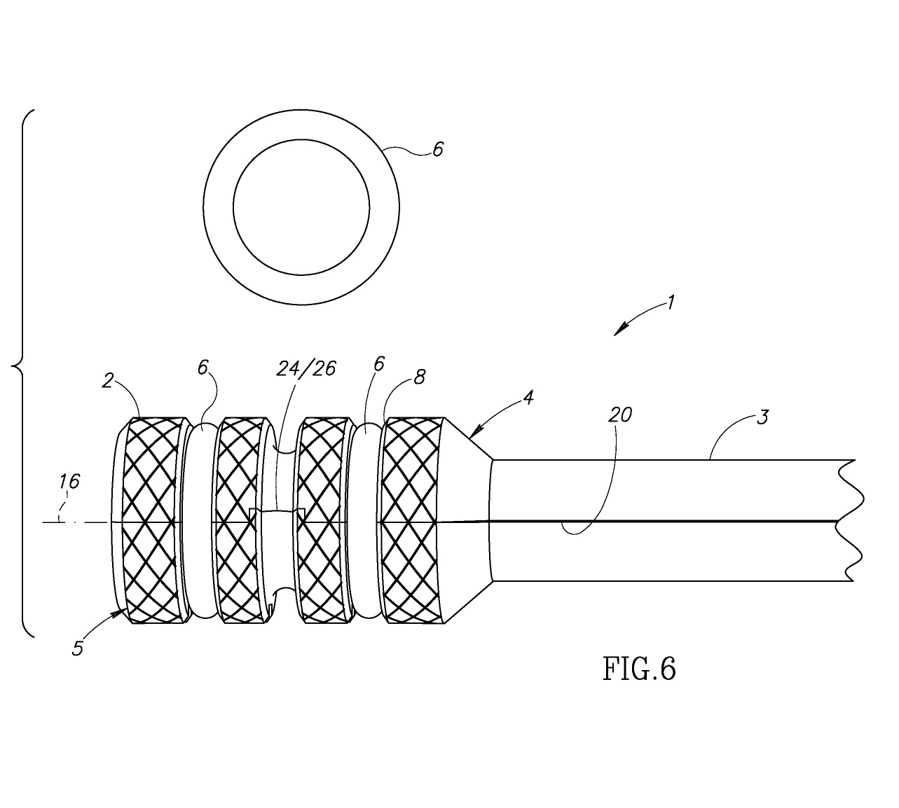

Referring to FIGS. 4 and 5, the illustrated instrument 1 is provided with alignment structure to properly align the instrument halves 4 and 5. The internal surfaces of the illustrated handle 2 is provided with a tongue and groove arrangement, such tongues 24 formed on the “tabbed” instrument half 5 and grooves 26 formed on the “notched” instrument half 4. Similarly, the cannula 3 is provided with tabs 28 on the tabbed instrument 5 and notches 30 on the notched instrument half 4. The tabs 28 and notches 30 are provided on the illustrated instrument 1 in a proximal set near the handle 2 and a distal set at the cannula tip 12. When the instrument halves 4 and 5 are joined, the tongues 24 engage the grooves 26, and the tabs 28 engage the notches 30 to thereby axially align the halves 4 and 5. FIG. 6 illustrates engagement of a tongue 24 with a groove 26 in the handle 2. Additionally, the distal set of the tabs 28 and notches 30 cooperate to prevent the halves of the cannula 3 from skewing misalignment.

While the grooves 26 and notches 30 are provided on the illustrated instrument half 4 and the tongues 24 and tabs 28 are illustrated as being provided on the instrument half 5, it is foreseen that the grooves 26, notches 30, tongues 24, and tabs 28 could be provided in cooperating sets on either of the instrument halves 4 and 5. Additionally, it is foreseen that a greater or lesser number of alignment structures or structure sets 22 could be provided. Finally, it is foreseen that other configurations of alignment structure 22 could be provided in the split endoscopic instrument 1.

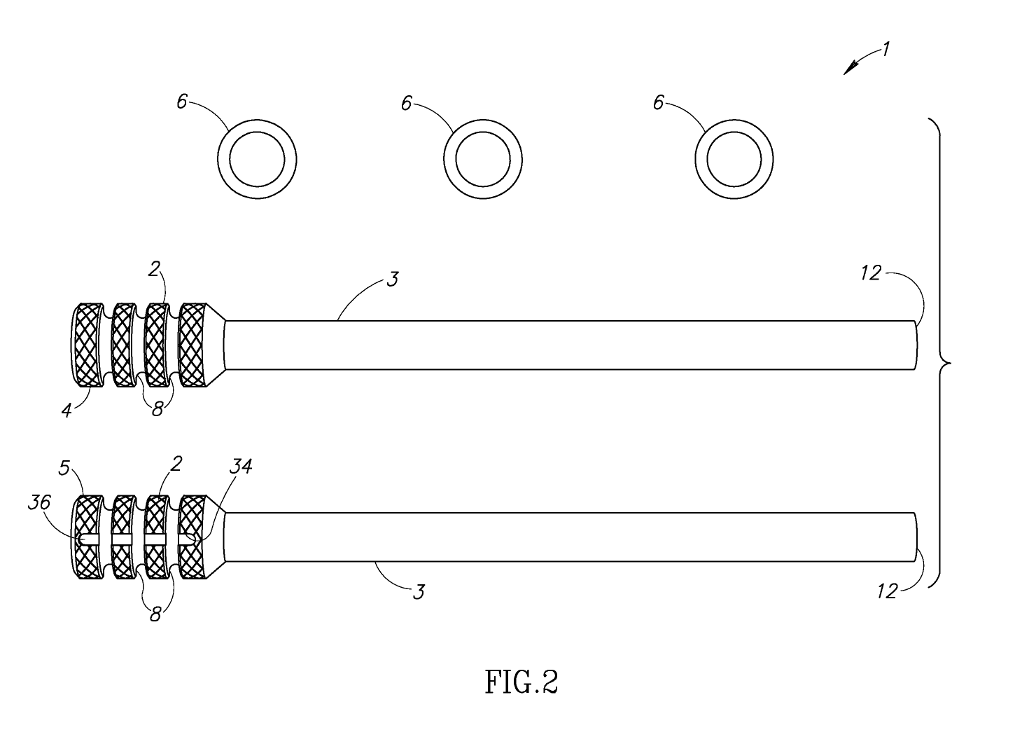

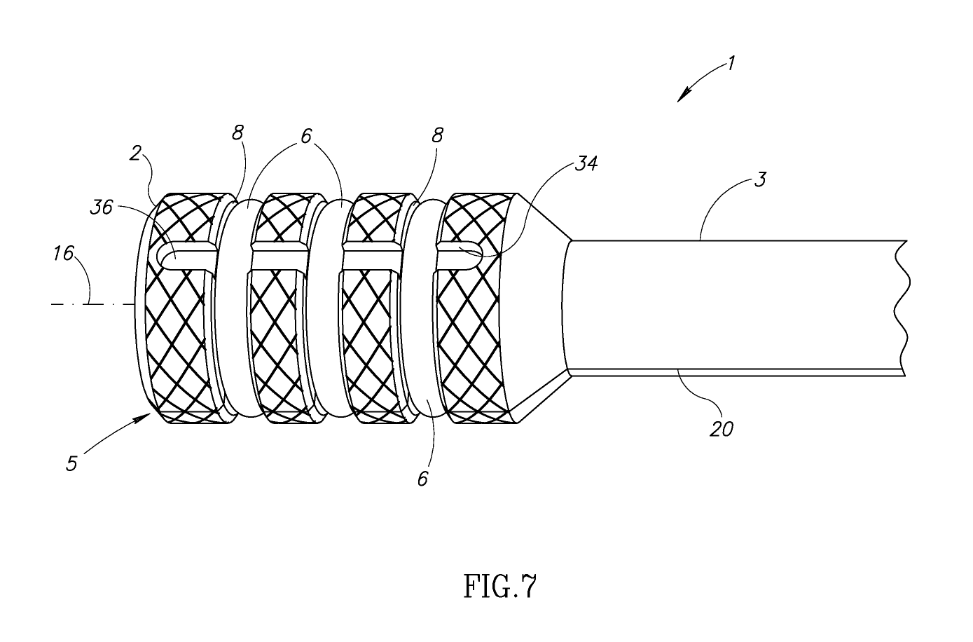

Referring to FIGS. 2 and 7, the illustrated retaining members 6 are O-rings which are formed of a somewhat flexible and elastic material, such as a suitable polymer. The O-rings 6 are stretched over the enlarged portions of the handle 2 and seated in the circumferential grooves 8 to retain the instrument halves 4 and 5 together. When it is desirable to separate the halves 4 and 5, the O-rings 6 can be slipped off the handle 2 to release the halves 4 and 5. Alternatively, the O-rings 6 can be quickly cut or severed, as with scissors, a scalpel, or the like. To facilitate either gripping the O-rings 6 for removal or cutting the O-rings 6, the illustrated handle 2 is provided with a recess 34 including a recessed anvil surface 36. The recess 34 provides clearance to slip a tool underneath the O-rings 6, either for removal or for cutting. The anvil surface 36 provides an alternative manner of cutting the O-rings 6 by simply slicing through the O-rings 6 along the recess 34 with the anvil surface 36 providing a support against which to cut the O-rings 6. While the illustrated instrument 1 is shown and described as having polymeric O-rings 6 functioning as retaining members, it is foreseen that other types of retaining members could be employed, such as various kinds of bands or releasable memory structures formed from materials other than polymers.

It is to be understood that while certain forms of the present invention have been illustrated and described herein, it is not to be limited to the specific forms or arrangement of parts described and shown.

What is claimed and desired to be secured by Letters Patent:

1. A split endoscopic instrument extending radially along a longitudinal axis aligned with a surgical site and transmitting surgical instruments having a dimension greater than a radius associated with said endoscopic instrument between a rear port and said surgical site, said split endoscopic instrument comprising:
a handle extending forwardly from said rear port to an elongated cannula in operative communication with said surgical site;
a plurality of circumferential grooves extending radially around said handle, said grooves being outwardly open;
a lumen adapted for reciprocal receipt of a portion of said surgical instrument through said rear port, handle and elongated cannula toward said surgical site;
a parting plane extending in opposite directions from said longitudinal axis through said endoscopic instrument thereby forming a first instrument half and a second instrument half;
said endoscopic instrument being operational between an open configuration and a closed configuration, said closed configuration corresponding to adjacently positioned, nonoverlapping instrument halves, said open configuration corresponding to non-adjacently positioned instrument halves; and
a plurality of interlocking connectors in communication with said retaining members for securing said endoscopic instrument, said interlocking connectors being spaced along said cannula and said handle and said plurality of retaining members being spaced along said handle at said circumferential grooves whereby said first instrument half being securely aligned to said second instrument half.

2. The split endoscopic instrument of claim 1 further comprising each of said instrument halves including a leading edge and a trailing edge, said edges extending along the intersection of said parting plane and said endoscopic instrument whereby said leading edges correspond and said trailing edges correspond to a location on said parting plan.

3. The split endoscopic instrument of claim 1 wherein said retainer members are O-rings, said split endoscopic instrument further comprising a recessed anvil surface presenting a recess bisecting said circumferential grooves for removal of said O-rings from said circumferential grooves.

4. The split endoscopic instrument of claim 1, which further comprises;
a pair of parted edges presented along the intersection of said parting plane with said endoscopic instrument on each of said instrument halves; and
a sealing means associated with said parted edges for hermetic sealing therealong.

5. The split endoscopic instrument of claim 1, which further comprises a side port communicating with said lumen and extending radially outward therefrom.

6. The split endoscopic instrument of claim 1 wherein the retainer members are comprised of materials other than polymers.

7. The split endoscopic instrument of claim 1 wherein said interlocking connectors comprise tongue and groove connecting means.

8. The split endoscopic instrument of claim 1 wherein said interlocking connectors comprise tab and notch connecting means.
